(19) United States
(12) Patent Application Publication
(10) Pub. No.: US 2026/0162817 A1
(43) Pub. Date: Jun. 11, 2026

(54) CONTROL OF INFORMATION FLOW, PRIORITIZATION AND MANIFESTATION OF DATA ASSOCIATED WITH AN ACTIVE HCP INTERACTION SPACE

(71) Applicant: Cilag GmbH International, Zug (CH)

(72) Inventors: Frederick E. Shelton, IV, Hillsboro, OH (US); Richard W. Timm, Cincinnati, OH (US); Andrew C. Deck, Cincinnati, OH (US); Jason L. Harris, Lebanon, OH (US)

(21) Appl. No.: 18/971,861

(22) Filed: Dec. 6, 2024

(51) Int. Cl. G16H 40/67; A61B 17/00

(52) U.S. Cl. CPC G16H 40/67; A61B 17/00; A61B 2017/00119

(57) ABSTRACT

A surgical system may include a processor configured to receive data from a medical instrument during a surgical procedure. The system may analyze the instrument data to determine the current surgical task being performed and may further reference surgical data from a database of prior procedures. Based on the analysis, the system may identify surgeon-specific preferences, the surgical context, or patient factors to adjust the level of information displayed on a screen. The system may prioritize critical data based on factors such as risk to the patient, task complexity, and the importance of specific information for documentation or decision-making.

### CROSS-REFERENCE TO RELATED APPLICATIONS

This application is related to the following, filed contemporaneously, the contents of each of which are incorporated by reference herein:
Attorney Docket No. END9638USNP1, entitled PROGRESSIVE ADVANCEMENT OF AUTOMATED LEVEL BASED ON LEARNED COMPLIMENTARY ASSISTANCE
Attorney Docket No. END9638USNP2, entitled ADJUSTING AUTOMATED COOPERATIVE OPERATIONS BASED ON SITUATIONALLY DERIVED CONSTRAINTS,
Attorney Docket No. END9638USNP3, entitled ASSISTANCE ADVANCEMENT MULTI-SYSTEM INTERACTION,
Attorney Docket No. END9638USNP4, entitled MONITORING AND IDENTIFYING SURGEON CONTROL AND SUGGESTING A TASK THAT MAY BE DONE AUTONOMOUSLY,
Attorney Docket No. END9638USNP6, entitled ADAPTIVE RETRACTION FORCE CONTROL,
Attorney Docket No. END9638USNP7, entitled ADJUSTMENT OR DISPLAY OF OPTIONS OF POSITIONAL OR ORIENTATION IMPLICATIONS ON SURGICAL TOOL USAGE, and
Attorney Docket No. END9638USNP8, entitled ADJUSTMENT OF PHYSIOLOGIC FUNCTION SUPPLEMENTATION CONTROL.

The contents of each of the following are incorporated by reference herein:
U.S. patent application Ser. No. 18/810,323 entitled METHOD FOR MULTI-SYSTEM INTERACTION, filed on Aug. 20, 2024;
U.S. patent application Ser. No. 18/960,006 entitled METHOD FOR SMART SURGICAL SYSTEMS filed on Nov. 26, 2024; and
U.S. patent application Ser. No. 18/954,186 entitled METHOD FOR MULTI-SYSTEM INTERACTION, filed on Nov. 20, 2024.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and improved technologies due to patient safety and a general desire for maintaining traditional practices.

### SUMMARY

A surgical system may include a processor configured to receive data from a medical instrument during a surgical procedure. The system may analyze the instrument data to determine the current surgical task being performed and may further reference surgical data from a database of prior procedures. Based on the analysis, the system may identify surgeon-specific preferences, the surgical context, or patient factors to adjust the level of information displayed on a screen. The system may prioritize critical data based on factors such as risk to the patient, task complexity, and the importance of specific information for documentation or decision-making.

The system may dynamically adapt the information displayed during the procedure to reduce cognitive load on the operator by prioritizing higher-risk information and deprioritizing less critical data. The display may be configured based on real-time input from the operator, allowing user preferences to influence the hierarchy of information shown. The system may compare the current procedure with (e.g., prior) procedures to identify disparities, generate recommendations for improving surgical techniques or device performance, and/or adjust the displayed information accordingly.

In examples, the system may incorporate patient-specific data, such as anatomy, comorbidities, and intraoperative measurements, to refine its prioritization of information. When the patient-specific data indicates heightened risk, the system may generate an alert, so the operator is (e.g., immediately) aware of critical conditions.

### BRIEF DESCRIPTION OF THE DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example surgical system that may include a surgical instrument.

FIG. 6 illustrates a smart system display that may provide an interface for presenting categories of information during a surgical procedure.

FIG. 7 illustrates a flow diagram for actions that may be performed by the smart system.

FIG. 8 illustrates a schematic representation of an AI/ML-enabled system framework that may perform actions related to surgical data processing and decision support.

### DETAILED DESCRIPTION

A more detailed understanding may be had from the following description, given by way of example in conjunction with the accompanying drawings.

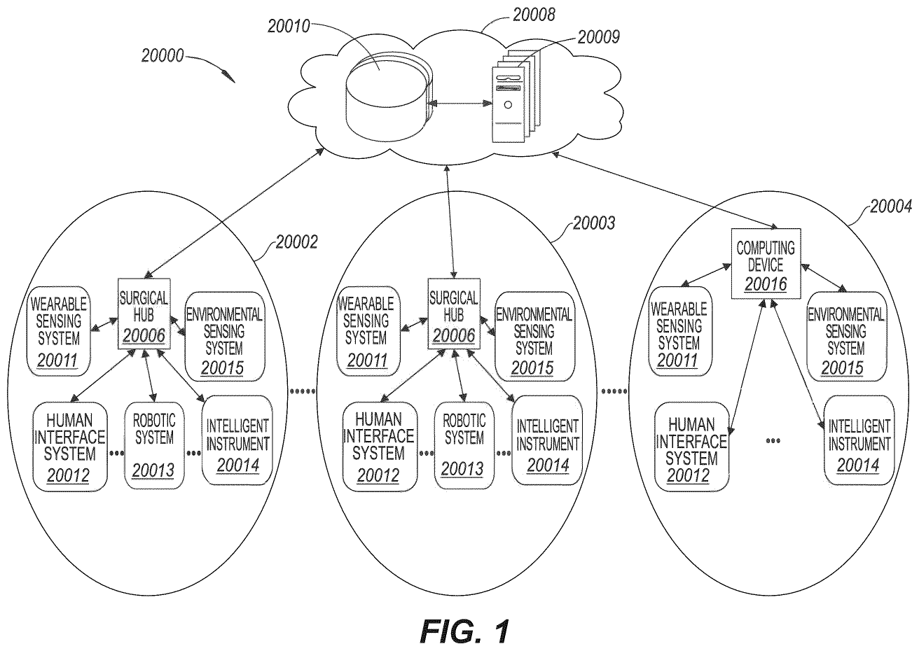

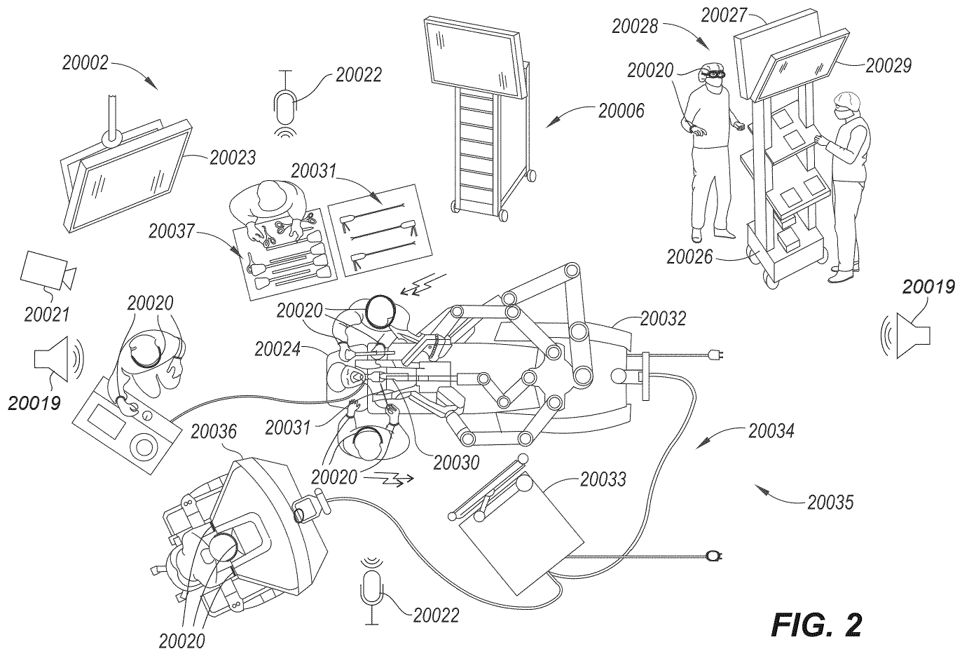

FIG. 1 shows an example computer-implemented surgical system 20000. The example surgical system 20000 may include one or more surgical systems (e.g., surgical sub-systems) 20002, 20003 and 20004. As described herein, a system may be referred to as systems (e.g., a collective system) For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, which is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

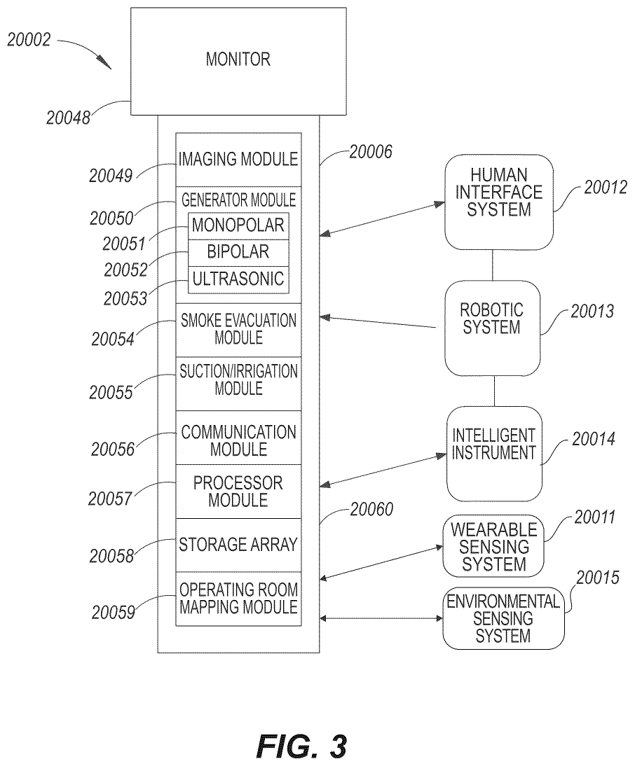

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

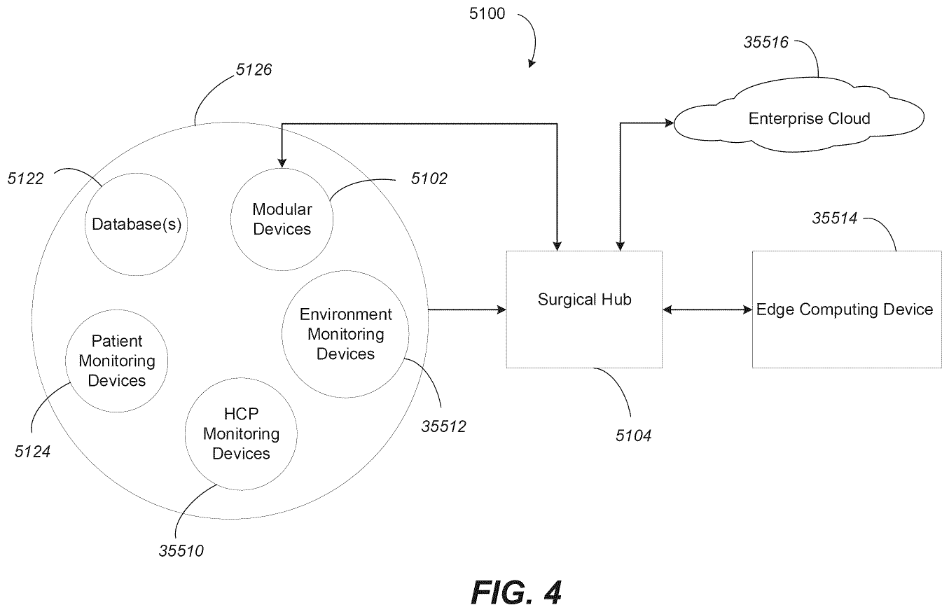

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

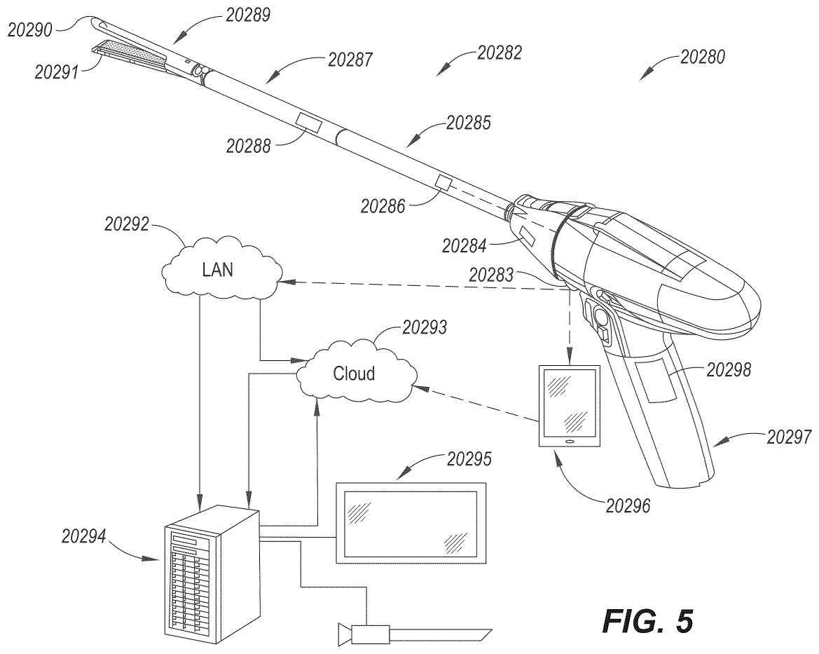

FIG. 5 illustrates an example surgical system 20280 that may include a surgical instrument 20282. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

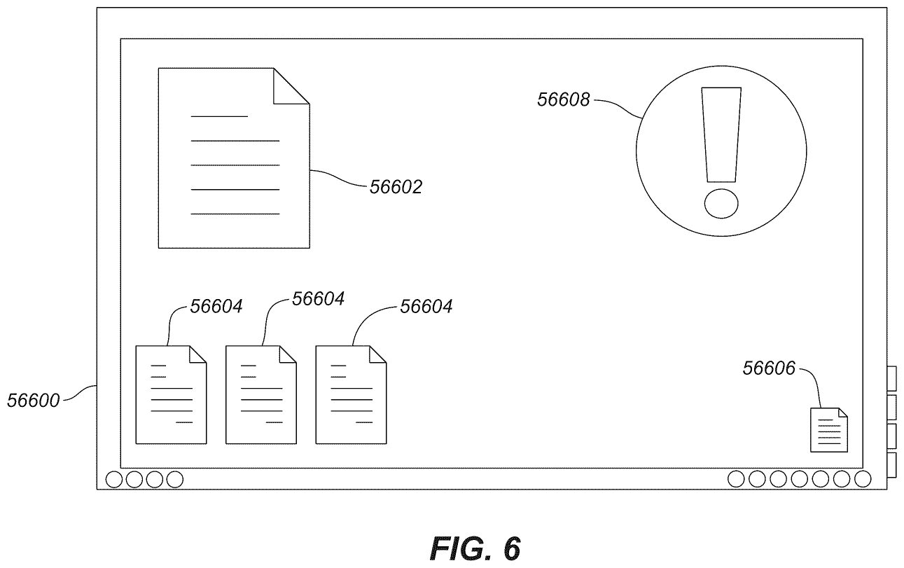

FIG. 6 illustrates a smart system display 56600 that may provide an interface for presenting categories of information during a surgical procedure. The display may adjust the visual prominence of information categories based on the context of the procedure, user preferences, and data relevance. For example, prioritized information 56602 may occupy a central or visible position, enabling its accessibility to the surgical team. Prioritized information may may correspond to data such as patient vitals or task-specific metrics that are associated with decision-making during, for example, high-risk phases of the procedure.

Standard priority information 56604 may be displayed alongside prioritized information and, for example, with reduced visual emphasis. Standard priority information may include procedural updates, instrument status, or medium-priority notifications that the surgical team may indirectly monitor. The system may utilize algorithms to determine which datasets fall into a standard priority category, balancing relevance with cognitive load to avoid overwhelming the user, so that contextual information remains accessible.

Deprioritized information 56606 may include data that, while not immediately relevant to the ongoing surgical task, may have utility for broader procedural oversight or post-procedural analysis. For example, the deprioritized information may include ergonomic feedback, secondary device performance metrics, or ongoing documentation tasks. The system may position such information in areas of the display that allow unobtrusive monitoring without detracting from higher-priority elements.

Alert 56608 may be visually distinct and designed to draw attention to significant or unexpected events during the procedure. Alert 56608 may correspond to anomalies, patient-specific risks, or instrument malfunctions that are associated with (e.g., immediate) intervention. The system may prioritize the alerts within the overall hierarchy of information, escalating their prominence based on severity, potential impact on the patient, or procedural phase.

The smart system display 56600 may support the management of information through adaptive configurations that align with the surgical workflow. The layout may change in response to user inputs, evolving procedural use cases, or pre-determined hierarchies established by the system. This adaptability be associated with a distribution of information across various stakeholders in the operating room, such as the surgeon, anesthesiologist, and supporting staff.

Control of the information flow, prioritization, and manifestation of data within the active HCP interaction space may include managing how data is displayed and escalated based on its context and relevance. The system may organize data according to the instruments in use, the tasks those instruments are performing, automated physical actions, or the outcomes the healthcare provider (HCP) is working to achieve. A hierarchy may dictate the level of display, which may be escalated or reduced based on factors such as the risk associated with patient data, the complexity of the task or device generating the data, or the importance of documenting or agreeing to the information. Relevance for technique or smart device performance may guide how information is presented.

To modify focus during laparoscopic and robotic procedures, the system may incorporate mechanisms to detect lapses in attention or suboptimal ergonomics. For example, a real-time display of a “mini avatar” on the laparoscopic monitor may help surgeons maintain awareness of their posture and movements. This visual feedback may serve as a reminder to correct body positioning or adjust their approach, thereby reducing fatigue and increasing longevity. By addressing both physical and cognitive factors, such tools may support surgeons in maintaining optimal performance throughout the procedure.

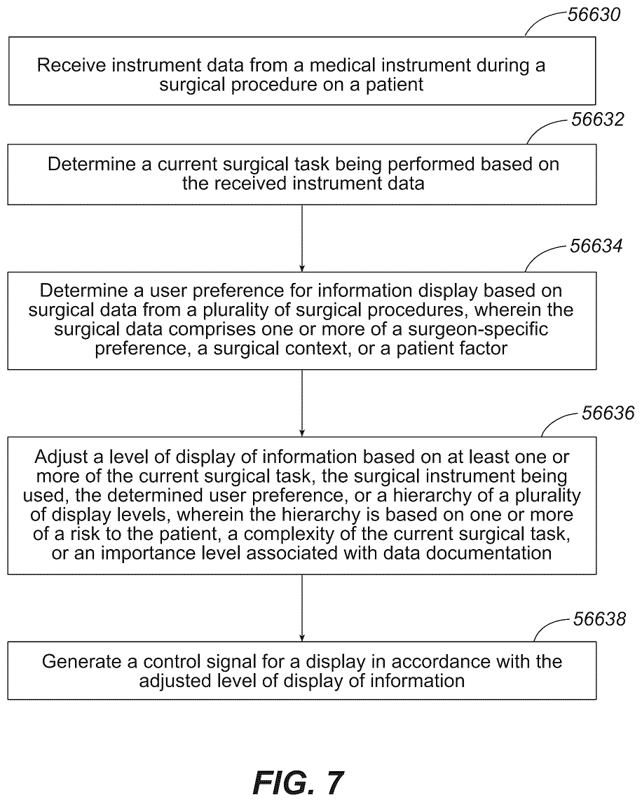

FIG. 7 illustrates a flow diagram for actions that may be performed by the smart system. At 56630, the system may receive instrument data from a medical instrument during a surgical procedure on a patient. At 56632, the system may determine a current surgical task being performed based on the received instrument data. At 56634, the system may determine a user preference for information display based on surgical data from a plurality of surgical procedures, and the surgical data may include one or more of a surgeon-specific preference, a surgical context, or a patient factor. At 56636, the system may adjust a level of display of information based on at least one or more of the current surgical task, the surgical instrument being used, the determined user preference, or a hierarchy of a plurality of display levels. The hierarchy may be based on one or more of a risk to the patient, a complexity of the current surgical task, or an importance level associated with data documentation. At 56638, the system may generate a control signal for a display in accordance with the adjusted level of display of information.

The level of display may be adjusted by prioritizing, on the display, information related to a higher risk to the patient over information related to a lower risk to the patient.

The level of display may be adjusted by deprioritizing, on the display, information related to a lower risk to the patient below information related to a higher risk to the patient. Deprioritizing the information may be associated with a reduced cognitive load on an operator of the surgical system.

The system may receive input from an operator of the surgical system during the surgical procedure indicating a preference for the level of display of information. The system may store the user preference for information display based on the received input.

The system may compare the adjusted level of display of information and the current surgical task with surgical data from the plurality of surgical procedures. The system may identify a disparity between the surgical procedure and a prior surgical procedure from the plurality of surgical procedures. The system may determine a recommendation for one or more of technique improvement or smart device performance modifications based on the identified disparity. The system may update the control signal to adjust the level of display of information to prioritize the determined recommendation.

The system may determine patient-specific data comprising including or more of a patient anatomy, a comorbidity, or an intraoperative physiological measurement. The system may update the hierarchy of the plurality of display levels to prioritize information relevant to the patient-specific data. The system may update the control signal to adjust the level of display of information based on the updated hierarchy, and the updated hierarchy may be associated with the level of display of information prioritizing the patient-specific data.

The system may determine that the patient-specific data indicates a heightened risk to the patient. The system may generate an alert based on the heightened risk to the patient, and prioritizing the patient-specific data may be associated with prioritizing the alert.

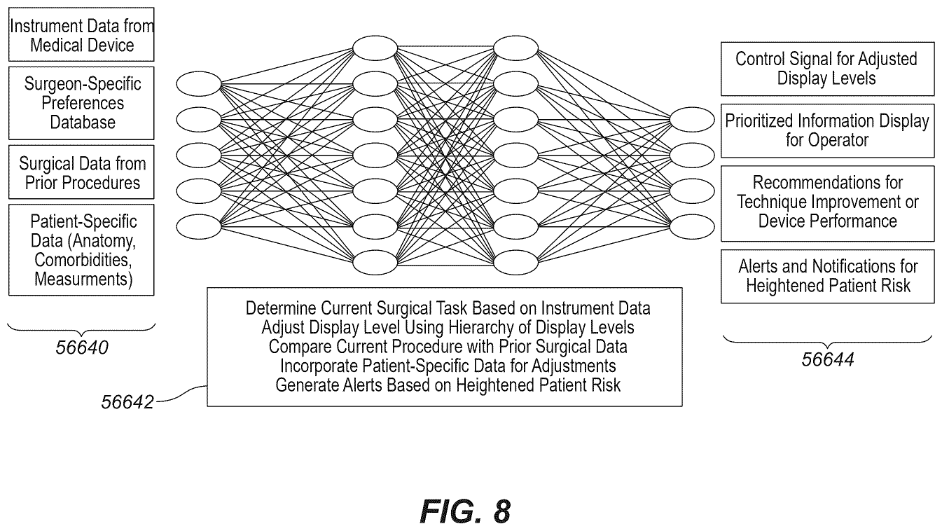

FIG. 8 illustrates a schematic representation of an AI/ML-enabled system framework that may perform actions related to surgical data processing and decision support. The framework may be organized as follows: input 56640, processing 56642, and output 56644. At input 56640, the system may receive data streams from sources, including instrument data generated by medical devices, surgeon-specific preferences stored in a database, historical surgical data from previous procedures, and patient-specific information such as anatomical details, comorbidities, or physiological measurements. The inputs may provide a foundation for the system to analyze the surgical environment and align processing and adjust outputs accordingly.

Information and data management within a surgical context may include organizing and directing data based on its source, destination, and relevance to the ongoing procedure. The system may handle subtypes of information, such as patient vitals, equipment status, and surgical progression metrics. Patient vitals may include (e.g., critical) parameters like heart rate and blood pressure, and equipment status may include current alarms and potential risks of future alarms. Surgical progression data may track the steps and milestones of the procedure, enabling the system to adapt its outputs accordingly.

The integration of in-situ devices with external imaging systems may allow instruments to serve multiple functions, such as marking anatomical structures or assisting with imaging overlays. For example, during laparoscopic or robotic procedures, instruments may be used to identify (e.g., critical) structures in semi-rigid organs like the liver or lung. Highlighting the structures may reduce the risk of inadvertent injury by providing real-time visual feedback and anatomical context to the surgical team. The ability to mark specific points on anatomical features using a handheld tool may enable precise triangulation when combined with CT scans or X-rays, for procedural accuracy.

Handheld tools may be leveraged to control settings on other smart devices, providing a centralized interface for managing disparate systems. For example, an instrument tip may be used to navigate a graphical user interface (GUI) displayed on a screen, enabling adjustments to device settings without interrupting the sterile workflow. This interaction may include tracking the instrument's movements or registering points within the field of view to modify system behavior. Such integration may affect procedural efficiency and reduce the cognitive load associated with managing multiple devices simultaneously.

Medical imaging data may be accessed and interacted with directly from the sterile field, allowing HCPs to manipulate preoperative scans during the procedure. For example, a device equipped with internal accelerometers or externally attached tracking components may communicate orientation and motion data to other systems. This capability may allow the instrument to act as a motion input device, facilitating real-time adjustments to 3D imaging or using the instrument as a virtual pointer to highlight specific regions on a digital display. Such features may be useful for analyzing anatomical variations or planning intraoperative strategies.

FollowMeMovement and position tracking technologies may support the functions by providing dimensional tracking and motion data from instruments lacking internal capabilities. The accelerometers may transmit data wirelessly to other systems, enabling monitoring and control of device motions. For example, such systems may detect tip movement, focus shifts, or auditory distractions to determine focus and procedural efficiency. The tools may serve as laser pointers for digital screens, affecting the surgeon's ability to interact with visual aids while remaining engaged in the sterile environment.

Monitoring the operating room (OR), staff, and surgical users may include tracking the procedure's progression, staff interactions, and available instruments to control the flow of information effectively. The system may leverage data about jobs, outcomes, and constraints (JOC), alongside the procedural plan or surgical steps, to dynamically adjust how information is prioritized. For example, changes in data flow prioritization may reflect the user's role, the task at hand, and the performance of instruments or workflows that appear to deviate from expectations.

The system may track surgical steps to monitor and measure the attainment of surgical objectives. Automated displays may update procedural checklists in real time for alignment with the predefined steps of the operation. For example, during laparoscopic or robotic surgeries, the system may provide visual prompts to guide the surgical team through the process, so that (e.g., critical) tasks are completed in the correct sequence. Efficiency monitoring tools may assess how smoothly the procedure progresses, identifying bottlenecks or delays that may inform process improvements.

Processing 56642 may include computations to determine the current surgical task based on instrument data, as well as to align display adjustments with the hierarchy of information priorities. Processing 56642 may include comparing real-time procedural data with historical surgical records to identify patterns, disparities, or potential areas for improvement. Patient-specific information may be integrated into the decision-making process, enabling the system to adjust its outputs to reflect individual patients, procedural phases, and operator preferences.

The hierarchy of display levels managed in processing 56642 may consider a risk level associated with patient data, the complexity of the surgical task, and the importance of documentation or technique adjustments. For example, higher-risk data such as vital signs or device anomalies may be escalated in prominence on the display, while lower-priority information may be filtered or routed to other stakeholders in the operating room. This stage may include the generation of recommendations for surgical technique or device performance based on historical and real-time data comparisons.

Technology may assist in decision support for surgical tasks to achieve primary objectives, such as gaining access to specific anatomy or performing a transection. Data flow control and prioritization may operate on interaction levels, which may be predefined by the system or navigated based on the escalation of tasks. For example, notifications may be categorized by importance, type, or source, so that (e.g., critical) datasets such as patient vitals (heart rate, blood pressure, oximetry) are given higher priority. Intermediate-priority data, such as tissue tension or impedance observable by the surgeon, may be handled differently from low-priority information, such as ergonomic feedback or reporting metrics.

In examples, the escalation and management of data may be context-sensitive, adapting to the procedural phase, the role of the HCP, and the instrument's activity level. By routing and prioritizing notifications appropriately, the system may enable the efficiency of surgical workflows while maintaining focus on the most relevant data for decision-making. For example, while ergonomic data may be useful for post-procedure analysis, it may not interrupt the surgeon's focus during (e.g., critical) tasks, and a balance between situational awareness and cognitive demands may be determined.

Notification buffering and prioritization may manage situations where the number of received or created notifications exceeds the available display capacity. Notifications may be buffered until they are acknowledged in some form. Acknowledgment methods may include automated acknowledgment, where messages are considered addressed after a specified timeout; manual acknowledgment, where users actively confirm receipt of notifications; or semi-automated acknowledgment, where the system detects secondary signals, such as eye-tracking, to determine user acknowledgment. Such a buffering may allow for a notification history to be created, enabling users to review prior messages for situational awareness or procedural analysis.

Sorting of buffered notifications may follow established prioritization methods. For example, notifications may be organized based on a first-in, first-out (FIFO) approach, which prioritizes messages in the order they were received. The system may apply a hierarchical priority model, where messages are sorted based on predefined importance levels. The sorting may enable the most relevant and urgent notifications are presented promptly, reducing the risk of information overload and enabling efficient decision-making during (e.g., critical) moments in the procedure.

Notification segregation and flow control may include routing messages to users based on their respective roles and responsibilities. For example, notifications about patient vitals may be directed to an anesthesiologist, and messages regarding equipment status may be routed to a circulating nurse or technician. Such segregation may minimize the burden of viewing unnecessary messages for users who do not require that information.

Notifications may be adjusted and manipulated based on coexisting messages and the procedural context. For example, if a low-priority notification regarding an ultrasonic scalpel issue is received simultaneously with a high-priority message about an insufflator, the system may deprioritize the scalpel notification. Instead of displaying the scalpel notification prominently, the system may provide an icon or indicator in a less prominent location, such as a corner of the surgeon's monitor, to signal the issue without distracting from the urgent insufflator message. Such may enable integration of multiple notifications and maintain clarity and focus of the displayed information.

The integration of notification buffering, sorting, segregation, and adjustment may enable a dynamic and context-sensitive information flow during surgical procedures. By aligning the presentation of notifications with the user's role and the procedural context, the system may determine the balance between situational awareness and cognitive load, so that (e.g., critical) outputs are not overlooked while managing the flow of secondary or less urgent information effectively.

The routing of information within the surgical room may depend on the context in which the data is generated and the intended recipients. For example, associated information may be directed based on the surgical line of sight and the user of the active device. In examples involving a smart connected endocutter, an alarm generated while the device is being reloaded by a scrub technician may not hold immediate relevance to the surgeon. The alarm may be routed to a staff monitor where the scrub technician can address the issue, leveraging geofencing or other contextual tools to determine the most appropriate recipient.

Information may be routed based on its type. Maintenance messages may be delivered to relevant personnel with tailored levels of detail. For example, a smart multi-output electrosurgical generator experiencing a damaged output may send a general alarm to the surgeon, indicating that the output is disabled. A detailed message may be directed to the maintenance team, specifying the device's serial number, the damaged output, and potential repairs. Alarms, such as a ground pad not being connected, may remain within the purview of the surgical staff in the room, as they are equipped to resolve such issues promptly.

Prioritization of information may include discriminating between competing data sources to identify the most relevant signal. Where multiple alarms arise simultaneously, prioritization may depend on the type of fault, the proximity of the device to the surgical zone, or the severity of the alarm. For example, if a handpiece and a generator report faults, the system may prioritize the generator if its error is likely to affect the handpiece's function. Devices physically closer to the patient or those with greater influence on the patient's condition may take precedence over those farther away or with less direct impact.

The prioritization process may further incorporate calculated indices that combine alarm severity with device criticality. A low-severity alarm from a high-criticality device may receive higher priority than a high-severity alarm from a low-criticality device. Such may allow the system to manage competing sources effectively, leveraging a database of error codes, contextual factors, and manual selections to determine the flow of information during the procedure. Devices that are actively involved in the patient's treatment, such as instruments and endoscopes, may be prioritized over less immediately relevant equipment, such as visualization monitors.

In examples, the system may include network management and interaction capabilities that alter data pipelines dynamically based on parameters such as the data source, the surgical context, or the intended destination. For example, the data stream pathways may be adjusted to prioritize patient-specific information during (e.g., critical) phases of surgery while routing less urgent data to supporting staff. Such may enable relevant information to be delivered to the right stakeholders at the right time, enhancing procedural efficiency and safety.

Technique evaluation and suggestion systems may serve as tools for the skills of healthcare providers, such as surgical residents. For example, learning centers may use recorded surgical cases to create debriefings or walkthroughs of user actions, comparing individual actions to outcomes. This paired comparison approach may accelerate the development of technical competence by identifying specific areas for improvement. External 2D cameras, combined with inertial measurement unit (IMU) data, may measure distractions, interruptions, or staff focus during procedures, such as tracking gaze or head position. These insights may provide actionable feedback to residents, enabling them to refine their skills over time.

Planning and problem-solving tools may focus on minimizing disruptions in the operating room, such as for nurses to leave the room to retrieve equipment. For example, external 2D cameras may track nurse movements and share data with an online application, generating a post-operative report that identifies inefficiencies in material and equipment management. This data may be used to determine pre-procedural planning, so that resources are readily available and reducing extended operating room time.

The system may support surgical objective attainment by offering decision support tools tailored to primary goals. These tools may include quality monitoring mechanisms, which assess outcomes in real-time or retrospectively to evaluate performance metrics. For example, during hemostasis interventions, the system may track instrument performance and tissue response. Efficiency monitoring tools may highlight opportunities to streamline workflows, such as reducing instrument exchange times or determining the placement of surgical tools to minimize disruptions.

Surgical workflow monitoring may include instrument tracking and the automated updating of checklists to reflect real-time progress. For example, the system may track the movement and angles of instruments to evaluate consistency in manipulation techniques. This data may provide insights into surgeon fatigue, which may contribute to errors. By analyzing manipulation angles relative to gravity or table height, the system may recommend ergonomic adjustments, such as rotating the patient or adjusting the table height, to reduce stress on the surgeon and affect procedural outcomes. Performance metrics may help identify discrepancies between a surgeon's technique and that of their peers, promoting consistency and longevity.

Interactive systems may assist with life-sustaining activities that support surgical care without directly achieving primary objectives. For example, the systems may monitor vital signs and endoscopic images to provide continuous feedback on the patient's condition. Anesthesia interventions may be supported by alerts or recommendations that help the anesthesiologist maintain physiological parameters. Interventions, such as managing fluid levels or monitoring devices (e.g., critical) to life support, may be integrated into the system's monitoring capabilities.

Determining operational control of instruments may include identifying the individual responsible for a specific device at a given moment. For example, in laparoscopic or robotic procedures, the system may associate instrument usage with individual operators, providing personalized post-operative performance analytics. The capability may differentiate metrics relevant to the attending surgeon from those associated with a resident or other team members. Identifying users may include wearable devices or input sequences, such as inertial measurement unit (IMU) taps, linked to specific personnel. The data points may support feedback and contribute to actionable post-operative analysis.

The system may assist with cognitive overhead tasks in surgical care by managing aspects of the procedure that do not directly contribute to primary objectives, such as transecting tissue or gaining anatomical access. The tasks may include life support, environmental management, and auxiliary decision-making processes, allowing healthcare providers to focus on (e.g., critical) surgical steps. By addressing the overhead responsibilities, the system may reduce the cognitive burden on surgical teams and streamline overall workflow.

Decision-making tools may affect the system's utility by supporting forecasting, risk assessment, and mapping of physiological systems. For example, the system may project potential outcomes based on various decision alternatives, offering weighted risk assessments to evaluate trade-offs. Mapping interrelated physiological systems may help the surgical team understand how displayed information relates to broader systemic functions. Projections of organ or system complications based on the current intraoperative situation may allow for proactive interventions. These tools may integrate overlays of comorbidities to refine decision-making, and post-procedure follow-up recommendations may enable continuity of care. Consideration of current instrument and staff availability may provide additional context, highlighting constraints or opportunities during (e.g., critical) decision points.

Management of the OR environment may include determining the availability and readiness of tools, equipment, and supplies. The system may monitor inventory and equipment status, verifying that instruments are present and functioning before the procedure begins. This may extend to real-time monitoring, alerting the surgical team to equipment that requests attention or replacement, thus minimizing disruptions and delays.

The system may play a role in mitigating distractions that can disrupt focus during surgical procedures. Thes distractions may include equipment failures, personnel entering or leaving the OR, and communication interruptions such as phone calls or pager notifications. By detecting and managing these disruptions, the system may help maintain a controlled environment. For example, IMUs and 2D cameras may be deployed to track noise levels, staff movements, and other distractions. Insights from these devices may be delivered as part of a post-operative report, providing data to refine future workflows.

Problem-solving capabilities within the system may include analyzing symptomatology, summarizing vital signs, and performing commonality analysis of previous outcomes. Symptomatology tracking may allow the system to correlate intraoperative findings with pre-existing patient conditions, providing insights into potential complications. Vital sign summation may present aggregated physiological data in an easily interpretable format, enabling rapid assessments of the patient's status. Commonality analysis may identify patterns across historical data, helping predict outcomes based on similar procedural contexts or patient profiles.

At output 56734, the system may produce a control signal that adjusts the display configuration, prioritizing relevant information to align with the surgical context. For example, data related to a heightened patient risk may trigger alerts that are prominently displayed, and non-urgent updates may be deprioritized or displayed to supporting staff, for example. The system may offer recommendations for device settings, technique modifications, or other procedural modifications based on identified disparities or inefficiencies. The output may be designed to provide actionable insights to the surgical team while maintaining a balance between cognitive load and situational awareness.

Emergency or rapid-response notifications may be prominent when unusual or unexpected datasets cannot be directly or indirectly observed by the surgeon. For example, such data may be routed to other HCPs not present in the operating room, which may be beneficial when surgical teams manage multiple simultaneous cases. Controlled escalation of data may depend on factors like occurrence, severity, risk, or magnitude of changes in the monitored variable. For example, dissection around (e.g., critical) vascular structures may trigger quicker and more prominent notifications compared to less urgent tasks such as gallbladder dissection.

An alert and/or notification may be an output 56644, with the system modifying the prominence thereof and routing based on severity and relevance. For example, a sudden drop in a physiological parameter may result in an immediate alert to the surgeon, while a less urgent issue such as a low battery warning for a secondary device may be directed to a circulating nurse or technician. The system may generate post-procedure recommendations or summaries that synthesize insights from the operation for future planning or analysis.

Highlighting unusual or unexpected data results may depend on the severity of the anomaly and its relevance to the ongoing procedure. Notifications may vary between major and minor alerts, with lower-severity data routed to other HCPs or support staff, such as anesthesiologists or scrub nurses, rather than the surgeon. For example, if pulse oximetry data is interrupted but other vitals remain stable, the notification may be directed to the anesthesiologist without alerting the surgeon. The system may anticipate root causes and suggest corrective actions based on prior user feedback, facilitating efficient issue resolution.

To reduce cognitive load on the surgeon, notifications may be minimized to focus on aspects of active functions. For example, notifications from instruments initialized but not yet required may be sent to non-surgeon HCPs, such as circulators. If an endoscope is activated prematurely during a laparoscopic anterior resection (LAR) and malfunctions, this information may be directed to a non-surgeon staff member rather than the surgeon. Once the instrument is no longer in use or being used, such as after visualizing an anastomosis, subsequent malfunctions may be routed to support staff or a sales representative for further action.

Recording and annotating surgical procedures may include linking notations and comments to a timeline of the procedure, enabling documenting relevant events and decisions. Such may integrate with the notification escalation system, so outputs are balanced while maintaining the visibility of (e.g., critical) information. Notification escalation may work by prioritizing messages based on their relevance, urgency, and the procedural context, while outputs remain available to the user.

Modification of information may depend on the intended recipient and the context of the notification. For example, while a surgeon may use a concise summary of an equipment issue, maintenance staff may benefit from detailed information, including error codes and repair instructions. The system may add, duplicate, or eliminate information to suit the recipient's role and responsibilities. This flexibility in data management may allow for seamless communication and coordination across the surgical team while maintaining focus on the most pertinent information.

Requesting assistance during surgical procedures may include notifications tailored to specific healthcare providers (HCPs) regarding support with a particular functional task. Such notifications may enable appropriate individuals to be alerted promptly, facilitating coordinated responses. For example, when an HCP requests assistance with a complex or unexpected task, the system may generate notifications directed at personnel, such as additional surgeons, anesthesiologists, or support staff, based on the nature of the assistance.

The system may issue notifications related to equipment or instruments, prioritizing these based on their relevance to the ongoing task. For example, if an instrument malfunction occurs mid-procedure, the system may highlight the issue to the circulating nurse or technician and suggest alternative tools. Notifications may incorporate prioritization levels, so that urgent needs, such as equipment for life-sustaining activities, are addressed promptly, while less (e.g., critical) requests may be queued or deprioritized.

Documentation in surgical procedures may include the integration of real-time and post-procedure data to affect team communication, medical reporting, and overall surgical outcomes. For example, medical reporting may focus on surgeon preferences for specific tasks, such as the firing of powered circular staplers during laparoscopic colorectal anastomoses, being accurately executed. By sensing device closure and tip movement, the system may communicate this data via Bluetooth to a central hub, so that factors like position and stability are determined. Such may help reduce the incidence of intraoperative positive air leak tests and postoperative anastomotic leaks.

Team communication during procedures may be supported by systems designed to record case videos for consistency in video documentation for laparoscopic procedures. This may include sensors detecting the introduction of instruments through a trocar, using Bluetooth to synchronize with a central hub. Instruments may feature indicators molded or printed onto their surfaces, which may pair with external seal housings to enable synchronization between internal cameras and external room cameras. This may enable movement of instruments and personnel in the operating room to be captured, facilitating the generation of a timeline of the procedure.

Coordination of recordings across multiple devices may allow for documentation of surgical workflows. External room cameras monitoring the operating room may track instrument introductions, removals, and usage patterns, while internal cameras align the movements with surgical activities. The coordination may result in a procedural timeline that can be utilized for post-operative review, training, and quality assurance. The ability to document such workflows may affect the traceability and accountability of surgical processes.

Post-surgical annotation and video-based recommendations may further support quality and learning. Human or AI-based systems may annotate recorded videos, providing localized recommendations for technique adjustments or procedural modifications. These insights may be synthesized from a complete procedural file and shared with the surgeon during a review session. User-based control of information flow within the surgical interaction space may allow individuals to enable or disable specific information displays, tailoring the interface to their unique preferences and roles. Relevant data may be highlighted appropriately for the user.

1. A surgical system comprising:
a processor configured to:
receive instrument data from a medical instrument during a surgical procedure on a patient;
determine a current surgical task being performed based on the received instrument data;
determine a user preference for information display based on surgical data from a plurality of surgical procedures, wherein the surgical data comprises one or more of a surgeon-specific preference, a surgical context, or a patient factor;
adjust a level of display of information based on at least one or more of the current surgical task, the surgical instrument being used, the determined user preference, or a hierarchy of a plurality of display levels, wherein the hierarchy is based on one or more of a risk to the patient, a complexity of the current surgical task, or an importance level associated with data documentation; and
generate a control signal for a display in accordance with the adjusted level of display of information.

2. The surgical system of claim 1, wherein the level of display is adjusted by prioritizing, on the display, information related to a higher risk to the patient over information related to a lower risk to the patient.

3. The surgical system of claim 1, wherein the level of display is adjusted by deprioritizing, on the display, information related to a lower risk to the patient below information related to a higher risk to the patient, and wherein deprioritizing the information is associated with a reduced cognitive load on an operator of the surgical system.

4. The surgical system of claim 1, wherein the processor is further configured to:
receive input from an operator of the surgical system during the surgical procedure indicating a preference for the level of display of information; and
store the user preference for information display based on the received input.

5. The surgical system of claim 1, wherein the processor is further configured to:
compare the adjusted level of display of information and the current surgical task with surgical data from the plurality of surgical procedures;
identify a disparity between the surgical procedure and a prior surgical procedure from the plurality of surgical procedures;
determine a recommendation for one or more of technique improvement or smart device performance enhancement based on the identified disparity; and
update the control signal to adjust the level of display of information to prioritize the determined recommendation.

6. The surgical system of claim 1, wherein the processor is further configured to:
determine patient-specific data comprising one or more of a patient anatomy, a comorbidity, or an intraoperative physiological measurement;
update the hierarchy of the plurality of display levels to prioritize information relevant to the patient-specific data; and
update the control signal to adjust the level of display of information based on the updated hierarchy, wherein the updated hierarchy is associated with the level of display of information prioritizing the patient-specific data.

7. The surgical system of claim 6, wherein the processor is further configured to:
determine that the patient-specific data indicates a heightened risk to the patient; and
generate an alert based on the heightened risk to the patient, wherein prioritizing the patient-specific data is associated with prioritizing the alert.

8. A method for a surgical system comprising:
receive instrument data from a medical instrument during a surgical procedure on a patient;
determine a current surgical task being performed based on the received instrument data;
determine a user preference for information display based on surgical data from a plurality of surgical procedures, wherein the surgical data comprises one or more of a surgeon-specific preference, a surgical context, or a patient factor;
adjust a level of display of information based on at least one or more of the current surgical task, the surgical instrument being used, the determined user preference, or a hierarchy of a plurality of display levels, wherein the hierarchy is based on one or more of a risk to the patient, a complexity of the current surgical task, or an importance level associated with data documentation; and
generate a control signal for a display in accordance with the adjusted level of display of information.

9. The method of claim 8, wherein the level of display is adjusted by prioritizing, on the display, information related to a higher risk to the patient over information related to a lower risk to the patient.

10. The surgical system of claim 8, wherein the level of display is adjusted by deprioritizing, on the display, information related to a lower risk to the patient below information related to a higher risk to the patient, and wherein deprioritizing the information is associated with a reduced cognitive load on an operator of the surgical system.

11. The method of claim 8, wherein the method comprises:
receiving input from an operator of the surgical system during the surgical procedure indicating a preference for the level of display of information; and
storing the user preference for information display based on the received input.

12. The method of claim 8, wherein the method comprises:
comparing the adjusted level of display of information and the current surgical task with surgical data from the plurality of surgical procedures;
identifying a disparity between the surgical procedure and a prior surgical procedure from the plurality of surgical procedures;
determining a recommendation for one or more of technique improvement or smart device performance enhancement based on the identified disparity; and
updating the control signal to adjust the level of display of information to prioritize the determined recommendation.

13. The method of claim 1, wherein the method comprises:
determining patient-specific data comprising one or more of a patient anatomy, a comorbidity, or an intraoperative physiological measurement;
updating the hierarchy of the plurality of display levels to prioritize information relevant to the patient-specific data; and
updating the control signal to adjust the level of display of information based on the updated hierarchy, wherein the updated hierarchy is associated with the level of display of information prioritizing the patient-specific data.

14. The method of claim 13, wherein the method comprises:
determining that the patient-specific data indicates a heightened risk to the patient; and
generating an alert based on the heightened risk to the patient, wherein prioritizing the patient-specific data is associated with prioritizing the alert.

15. A surgical system comprising:
a processor configured to:
receive instrument data from a medical instrument during a surgical procedure on a patient;
determine a current surgical task being performed based on the received instrument data;
determine a user preference for information display based on surgical data from a plurality of surgical procedures, wherein the surgical data comprises one or more of a surgeon-specific preference, a surgical context, or a patient factor; and
adjust a level of display of information based on at least one or more of the current surgical task, the surgical instrument being used, the determined user preference, or a hierarchy of a plurality of display levels, wherein the hierarchy is based on one or more of a risk to the patient, a complexity of the current surgical task, or an importance level associated with data documentation; and

16. The surgical system of claim 15, wherein the level of display is adjusted by prioritizing, on the display, information related to a higher risk to the patient over information related to a lower risk to the patient.

17. The surgical system of claim 15, wherein the level of display is adjusted by deprioritizing, on the display, information related to a lower risk to the patient below information related to a higher risk to the patient, and wherein deprioritizing the information is associated with a reduced cognitive load on an operator of the surgical system.

18. The surgical system of claim 15, wherein the processor is further configured to:
receive input from an operator of the surgical system during the surgical procedure indicating a preference for the level of display of information; and
store the user preference for information display based on the received input.

19. The surgical system of claim 15, wherein the processor is further configured to:
compare the adjusted level of display of information and the current surgical task with surgical data from the plurality of surgical procedures;
identify a disparity between the surgical procedure and a prior surgical procedure from the plurality of surgical procedures;
determine a recommendation for one or more of technique improvement or smart device performance enhancement based on the identified disparity; and
generate a control signal to adjust the level of display of information to prioritize the determined recommendation.

20. The surgical system of claim 15, wherein the processor is further configured to:
determine patient-specific data comprising one or more of a patient anatomy, a comorbidity, or an intraoperative physiological measurement;
update the hierarchy of the plurality of display levels to prioritize information relevant to the patient-specific data; and
update the control signal to adjust the level of display of information based on the updated hierarchy, wherein the updated hierarchy is associated with the level of display of information prioritizing the patient-specific data.
